(19) United States
(12) Patent Application Publication
(10) Pub. No.: US 2015/0239949 A1
(43) Pub. Date: Aug. 27, 2015

(54) GENETICALLY MODIFIED MICROORGANISMS

(71) Applicant: Robert J. Hariri, Benardsville, NJ (US)

(72) Inventor: Robert J. Hariri, Benardsville, NJ (US)

(21) Appl. No.: 14/557,415

(22) Filed: Dec. 1, 2014

Related U.S. Application Data
(60) Provisional application No. 61/910,276, filed on Nov. 29, 2013.

(51) Int. Cl. C07K 14/495; A23L 1/305

(52) U.S. Cl. CPC C07K 14/495; A23L 1/3058; A23V 2002/00

(57) ABSTRACT

Embodiments of the present invention are generally related to genetically modified microorganisms. More specifically, embodiments of the present invention relate to genetically modified microorganisms having the avian follistatin genome engineered therein to create a microorganism strain having the enhanced properties of follistatin. In accordance with one embodiment of the present invention, a method of treating an individual with a muscle degenerative disease comprises administering an effective amount of avian follistatin, wherein the avian follistatin is derived from a genetically modified microorganism.

### CROSS-REFERENCE TO RELATED APPLICATIONS

This application is a continuation-in-part of U.S. patent application Ser. No. 11/750,128, filed May 17, 2007, entitled “Avian Follistatin Product,” which claims priority to U.S. Provisional Patent Application Ser. No. 60/801,266, filed May 18, 2006, the disclosures of which are incorporated herein by reference in their entireties. This application also claims priority to U.S. Provisional Patent Application Ser. No. 61/910,276, filed Nov. 29, 2013, the disclosure of which is also incorporated herein by reference in its entirety.

### BACKGROUND

1. Field of the Invention

Embodiments of the present invention are generally related to genetically modified microorganisms. More specifically, embodiments of the present invention relate to genetically modified microorganisms having the avian follistatin genome engineered therein to create a microorganism strain having the enhanced properties of follistatin.

2. Description of the Related Art

Tremendous progress in the genetic engineering of microorganisms, and in particular, yeast, has been achieved at the end of 20th century. Such achievements have included the complete genome sequence, genome-wide gene expression profiling, and whole gene disruption strains of yeast, among other achievements. However, despite such advances, the commercial use of such genetically-modified (GM) yeasts is still quite rare.

The protein known as avian follistatin, and in particular, chicken follistatin, has been identified as a very beneficial protein for human consumption as it pertains to the health of lean muscle tissue. Such benefits are described in part in commonly owned United States Patent Application Publication No. 2007/0275036, the disclosure of which is incorporated herein by reference in its entirety. However, like any product derivative of egg, there are a variety of health concerns associated therewith.

Thus, there is a need for genetically modified microorganisms having the avian follistatin genome engineered therein to create a microorganism strain having the enhanced properties of follistatin.

### SUMMARY

Embodiments of the present invention are generally related to genetically modified microorganisms. More specifically, embodiments of the present invention relate to genetically modified microorganisms having the avian follistatin genome engineered therein to create a microorganism strain having the enhanced properties of follistatin.

In accordance with one embodiment of the present invention, a method of treating an individual with a muscle degenerative disease comprises administering an effective amount of avian follistatin, wherein the avian follistatin is derived from a genetically modified microorganism.

### DETAILED DESCRIPTION

It is understood that the embodiments of the present invention are not limited to the particular methodologies, protocols and the like, described herein as they may vary. It is also to be understood the terminology used herein is used for the purpose of describing particular embodiments only and not intended to limit the scope of the present invention. Unless defined otherwise, all technical and scientific terms used herein have the same meaning as commonly understood by one of ordinary skilled in the art to which this invention belongs.

As utilized herein, the term “microorganism” is intended to be inclusive of any single cell or multicellular microscopic organism. In particular, the term microorganism shall include all of the prokaryotes, namely the bacteria and archaea; and various forms of eukaryotes, comprising the protozoa, fungi, algae, microscopic plants (e.g., green algae), and animals such as rotifers and planarians.

Many exemplary embodiments of the present invention pertain to yeast, and in particular the species Saccharomyces cerevisiae (“S. cerevisiae”) which is common form of yeast used in baking, winemaking, and brewing. S. cerevisiae is often termed a model organism because it scores favorably on a number of criteria. For example: as a single celled organism S. cerevisiae is small with a short generation time; the ability to grow S. cerevisiae as a haploid simplifies the creation of gene knockouts strains; S. cerevisiae can be transformed allowing for either the addition of new genes or deletion through homologous recombination; and, as a eukaryote, S. cerevisiae shares the complex internal cell structure of plants and animals without the high percentage of non-coding DNA that can confound research in higher eukaryotes. Each of these attributes of S. cerevisiae make it a suitable microorganism for embodiments of the present invention.

It is well known that S. cerevisiae was the first eukaryotic genome that was completely sequenced. The genome is composed of about 12,156,677 base pairs and 6,275 genes, compactly organized on 16 chromosomes. However, many researchers believe only about 5,800 of these are believed to be true functional genes. Given the amount of research and information available regarding yeast, and various strains and species thereof, identifying the proper locations to add and/or delete genes within the strains to create a genetically modified microorganism as described herein.

For purposes of embodiments of the present invention, any methods of genetically modifying microorganisms may be utilized to achieve the results desired with embodiments of the present invention. Accordingly, any known method of genetically modifying microorganisms shall be acceptable for various embodiments herein and should be deemed incorporated by reference herein.

In accordance with embodiments of the present invention, microorganisms may be genetically modified to include the genome for avian follisatatin, such that avian follistatin may be effectively grown within such microorganisms. A detailed description of avian follistatin may be found within commonly owned U.S. patent application Ser. No. 11/750,128, the scope of which is incorporated herein by reference in its entirety.

In one embodiment, yeast may be utilized to facilitate the growth of avian follistatin. By utilizing yeast, the resulting modified yeast may be utilized in a manner similar to traditional yeast. That is, various food products such as bread, pastries or other baked goods may be created having the avian follistatin protein therein.

Similarly, various fermented beverages, such as wine, beer or liquor may be created also having the avian follistatin protein therein. While certain measures may be implemented to preserve the integrity of the protein during the baking or fermenting process, the resulting product(s) will comprise an evenly distributed amount of the avian follistatin protein as opposed to adding the protein during the process, which may create highly concentrated areas of the protein.

Once such products comprising avian follistatin are created, they may be consumed and yield substantially the same benefits as those described in U.S. patent application Ser. No. 11/750,128, the scope of which is incorporated herein by reference in its entirety. As such, methods for treating an individual with a muscle degenerative condition may incorporated any number of the products described herein.

While the foregoing is directed to embodiments of the present invention, other and further embodiments of the invention may be devised without departing from the basic scope thereof. It is also understood that various embodiments described herein may be utilized in combination with any other embodiment described, without departing from the scope contained herein.

What is claimed is:

1. A method of treating an individual with a muscle degenerative disease comprising:
administering an effective amount of avian follistatin, wherein the avian follistatin is derived from a genetically modified microorganism.

2. The method of claim 1, wherein the step of administering the effective amount of avian follistatin comprises providing for consumption, at least one of a food product or a fermented beverage product.

3. The method of claim 2, wherein the food product comprises one of bread, pastry or baked goods.

4. The method of claim 2, wherein the fermented beverage product comprises one of wine, beer or liquor.
